(19) United States
(12) Patent Application Publication
(10) Pub. No.: US 2009/0118579 A1
(43) Pub. Date: May 7, 2009

(54) MEDICAL CAPSULE

(75) Inventor: Guenter DUERSCHINGER, Fuerth (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS MEDICAL SYSTEMS CORP., Tokyo (JP)

(21) Appl. No.: 12/265,409

(22) Filed: Nov. 5, 2008

Related U.S. Application Data
(63) Continuation of application No. PCT/JP2007/059367, filed on May 2, 2007.

(30) Foreign Application Priority Data
May 5, 2006 (DE) 10 2006 021 016.6
Mar. 11, 2007 (DE) 10 2007 011 556.5
Apr. 5, 2007 (DE) 10 2007 016 527.9

(51) Int. Cl. A61B 1/04; A61K 9/52; A61K 49/04

(52) U.S. Cl. 600/109; 424/457; 424/9.4; 424/9.41

(57) ABSTRACT

A medical capsule having a capsule configuration of size permitting introduction in a body, adapted to be at least partially dissolved over time by remaining in the intestines for a predetermined time or more. The medical capsule includes an inner casing provided at both ends with a discharge outlet and, surrounding the periphery thereof, an outer casing with a substantially uniform thickness. The inner casing and the outer casing are made of a material dissolved in the digestive secretion of the intestines. When the medical capsule remains in the intestines by, for example, entering a narrow area of the intestines, the wall of the outer casing begins to be dissolved. By this dissolution, the inner space of the inner casing communicates through the discharge outlet with the outside of the medical capsule. Consequently, a filling, such as a dye, medicine, etc., of the inner casing flows into the intestines.

### CROSS-REFERENCE TO RELATED APPLICATIONS

This application is a continuation of PCT international application Ser. No. PCT/JP2007/059367 filed on May 2, 2007 which designates the United States, incorporated herein by reference, and which claims the benefit of priority from Germany Patent Applications No. 10 2006 021 016.6, filed on May 5, 2006; No. 10 2007 011 556.5, filed on Mar. 11, 2007; and No. 10 2007 016 527.9, filed on Apr. 5, 2007, incorporated herein by reference.

### BACKGROUND OF THE INVENTION

1. Field of the Invention

The present invention relates to a dispensing apparatus and an in-duct bubble presence determining method in the dispensing apparatus.

2. Description of the Related Art

Video capsules for examining an intestinal tract for bleeding, inflammations, cancer etc. are described in United States Patent Publication Nos. 2001/0035902, 2002/0103417, U.S. Pat. Nos. 4,278,077 and 6,240,312, European Patent Application Publication No. 1604606, International Publication No. 03/005887, and Germany Utility-model No. 202004 020433.

For example, FIG. 4 in International Publication No. 03/005887 shows a device 50 useful for testing patency of the intestinal tract. This device 50 has a needle-shaped radio frequency identifier (denoted by 53) 2 mm in diameter and 12 mm tall coated with a glass, which is not dissolved by the digestive secretions of the intestinal tract. Accordingly, the device 50 needs to pass through a bottleneck existing in the intestinal tract before being excreted naturally.

Germany Utility-model No. 202004 020433 describes a capsule having a needle-shaped casing 2 made of a material resistant to all digestive secretions of human body.

In the course of endoscopical treatment or examination often a colouring of the upper and lower gastrointestinal tract (GI) is performed, for example with indigocarmine, because such colouring improves visibility of the inner intestine wall and the mucosa to determine vessel disease or other structures.

### SUMMARY OF THE INVENTION

An aspect according to the present invention is a medical capsule, having a capsule shape and size capable of being introduced into a subject body, at least a part of the medical capsule being dissolved over time by remaining in a predetermined time or more in a lumen. The medical capsule includes a casing, for forming the capsule shape, having at least two thin portions, wherein a communication opening for communicatively connecting an inner space of the casing to an outside of the medical capsule is formed when the thin portion is eliminated earlier than other portions of the medical capsule by dissolution of the casing.

Another aspect according to the present invention is a medical capsule, having a capsule shape and size capable of being introduced into a subject body, at least a part of the medical capsule being dissolved over time by remaining in a predetermined time or more in a lumen. The medical capsule includes a casing, for forming the capsule shape, having a thin portion; and a filling with which an inner space of the casing is filled, wherein the filling is discharged to an outside of the medical capsule after dissolution of the thin portion, with an portion of the casing other than the thin portion keeping substantially the capsule shape.

Still another aspect according to the present invention is a method for testing patency of an intestinal tract with a medical capsule having a capsule shape and size capable of being introduced into a subject body, at least a part of the medical capsule being dissolved over time by remaining in a predetermined time or more in a lumen. The method includes using as the medical capsule a capsule in which at least two thin portions of a casing forming the capsule shape is dissolved after a predetermined time to form a communication opening for communicatively connecting an inner space of the casing to an outside of the medical capsule, and the capsule keeps the capsule shape further after a predetermined time.

Still another aspect according to the present invention is A method for testing patency of an intestinal tract with a medical capsule having a capsule shape and size capable of being introduced into a subject body, at least a part of the medical capsule being dissolved over time by remaining in a predetermined time or more in a lumen. The method includes using as the medical capsule a capsule, the capsule including a casing, for forming the capsule shape, having a thin portion; and a filling with which an inner space of the casing is filled, wherein the filling is discharged to an outside of the capsule after dissolution of the thin portion, with an portion of the casing other than the thin portion keeping substantially the capsule shape.

Still another aspect according to the present invention is a method for treating an intestinal tract with a medical capsule having a capsule shape and size capable of being introduced into a subject body, at least a part of the medical capsule being dissolved over time by remaining in a predetermined time or more in a lumen. The method includes using as the medical capsule a capsule, the capsule including a casing, for forming the capsule shape, having a thin portion; and a filling with which an inner space of the casing is filled, wherein the filling is discharged to an outside of the capsule after dissolution of the thin portion, with an portion of the casing other than the thin portion keeping substantially the capsule shape.

Still another aspect according to the present invention is a medical capsule, having a capsule shape and size capable of being introduced into a subject body, at least a part of the medical capsule being dissolved over time by remaining in a predetermined time or more in a lumen. The medical capsule includes a casing, for forming the capsule shape, having an promptly-dissolving portion, wherein a communication opening for communicatively connecting an inner space of the casing to an outside of the medical capsule is formed when the promptly-dissolving portion is eliminated earlier than other portions of the promptly-dissolving portion by dissolution of the promptly-dissolving portion.

Still another aspect according to the present invention is a medical capsule for testing patency of an intestinal tract or treating an intestinal tract. The medical capsule includes a first state of dimension such that the capsule passes through an intestinal tract having no narrow area but is stopped by a narrow area; a second state of dimension such that the capsule can pass the narrow area of the intestinal tract; and an indicator material covered by a portion of the capsule hindering the indicator material to flow into the intestine in the first state and flows into the intestine in the second state. The portion of the capsule is dissolvable by secretions of the intestine after a predetermined time to allow the indicator material to flow into the intestine.

Still another aspect according to the present invention is a medical capsule for coloring an intestinal wall. The medical capsule includes a portion which is non-dissolvable by secretions of a stomach but which is dissolvable by secretions of an intestine after a predetermined time; and a dye covered by the portion in the medical capsule, not flowing out of the capsule when the portion is not dissolved but flowing out of the capsule when the portion is dissolved.

Still another aspect according to the present invention is a system including a video capsule for imaging an intestinal tract; and a medical capsule according to the present invention.

The features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a medical capsule with an inner casing having bores at both ends and an outer casing arranged around the inner casing;

FIG. 2 is a medical capsule with a casing formed by connecting an upper portion and lower portion and having thin wall portions at both ends;

FIG. 3 is a medical capsule with an inner casing having bores at both ends and an outer casing formed by connecting with two cup-shaped members and having thin wall portions at both ends;

FIG. 4 is a medical capsule formed by a hollow body and a continuous tube inserted into the hollow body;

FIG. 5 is a medical capsule formed by a hollow body with bores at both ends;

FIG. 6 is a medical capsule formed by a hollow body having a through-bore extending over the entire length; and

FIG. 7 is a medical capsule formed by a hollow body having bores at both ends with plugs closing the bores.

### DETAILED DESCRIPTION OF THE PREFERRED EMBODIMENTS

Exemplary embodiments of a medical capsule according to the present invention will be described in detail below with reference to the drawings. The invention is not limited to the embodiments.

A basic concept of a first embodiment is to provide a medical capsule which is chemically activated. The medical capsule has a capsule shape and size capable of being introduced into the subject body. The medical capsule also has at least an inner casing with two discharge outlets (openings) facing each other and at least a sealed outer casing. The inner and outer casings are made of a material which is dissolved after a predetermined time by digestive secretions of the intestinal tract (lumen). The inner casing is filled with a filling. The filling can flow from the discharge outlets into the intestinal tract following the dissolution of the outer casing no matter which side of the capsule arrives at a bottleneck. Due to the arrangement of the facing discharge outlets, the discharge ability of the filling is preferably influenced by the contraction of the small intestine. The filling may consist of, for example, granulated material, powder, gel or liquid.

An example of the filling is billowing bubble generated when the filling is discharged in the intestinal tract. The bubble passes through the bottleneck or the narrowed area (hereinafter, “narrowed area” includes “bottleneck”) and an intestinal tract without staying there, and is then excreted from the anus. Another example of the filling is gas with an exceptionally striking smell generated when it is discharged in an intestinal tract. This gas immediately passes through the narrowed area without staying there like wind, and flows out from the anus through the intestinal tract. As still another example, the filling may be discharged as a gel or a liquid which dyes the stool with a particularly striking colour. Alternatively, when the intestinal tract is empty, the filling may be naturally excreted in the form of a liquid dyed with a particularly striking colour.

Due to these characteristics, it is certainly acknowledged that the capsule has stuck at a narrow area, and that the outer casing has dissolved completely or partially. The inner casing remains at a narrow-area for a predetermined time until it is completely dissolved. The position of the inner casing can be determined using X-ray technology if a contrast medium such as barium sulphate is homogeneously-mixed and added to the material of the inner casing. A painkiller or other medicine may be added into the filling in the capsule.

As another embodiment, a medical capsule may be formed from only one filled casing made of the same material as the inner casing described above. This casing includes a lower portion and an upper portion which are hermetically joined to each other. The wall thickness of the casing is designed to thin by shaping the interior. As a result, the walls (thin wall portions) dissolve earlier than other portions, and the filling can flow into the intestinal tract. The emptied casing remains stuck at the narrow area for a predetermined time until it is completely dissolved. The location of the casing can be detected with X-ray technique if a contrast medium, for example, is homogeneously-mixed and added to the material of the casing.

If the capsule is naturally excreted from the intestinal tract without being dissolved and with keeping the original shape, the intestinal tract has patency which allows administration of the video capsule as first mentioned.

If a narrow area exists, the condition and the position of the bottleneck in especially the small intestine can be assessed and identified prior to operation, with a double balloon endoscope as described in Germany Patent Application Publication No. 10 2004 023 457.

Materials with predetermined dissolution times are sufficiently well-known in the pharmaceutical industry. Thus, for example, a capsule for treating “Crohn's disease” dissolves precisely in the “ileocecal region” after passing the stomach, the duodenum and the small intestine. And then, the capsule discharges the medicine “mesalazin” for treatment.

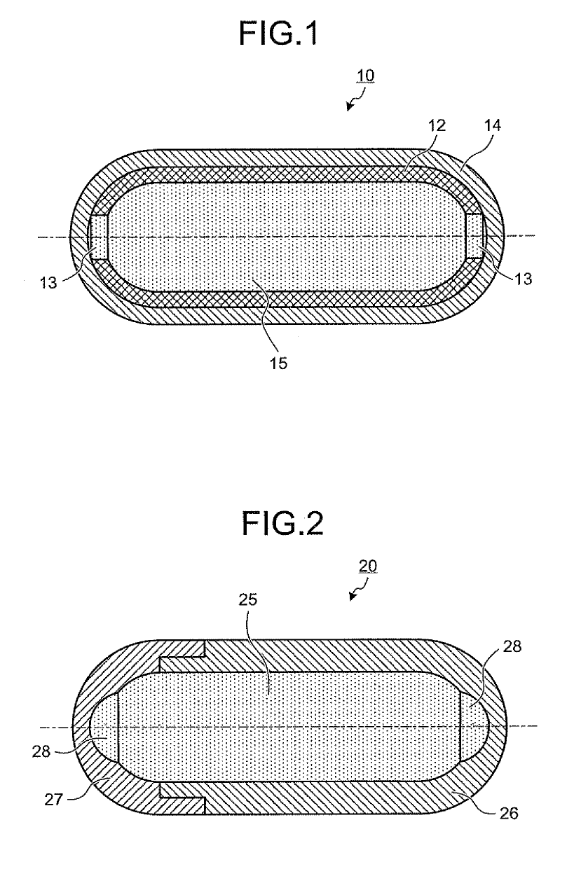

An example of the first embodiment will be described below with reference to FIGS. 1 to 3. FIG. 1 shows a medical capsule 10 for the penetrability testing of the intestinal tract. The medical capsule 10 includes an inner casing 12 which has two discharge outlets 13 which face each other, and a sealed outer casing 14. The inner casing 12 and the outer casing 14 are made of a material which is dissolved by the digestive secretions of the intestinal tract. The inner casing 12 is filled with a filling 15 of a granulated material, powder, gel or liquid. The outer casing 14 surrounds an outer surface of the inner casing 12 and has a substantially uniform thickness. The inner casing 12 has an inner space similar to the outline of the inner casing 12 and/or the outer casing 14.

FIG. 2 shows a medical capsule 20 for testing patency of the intestinal tract. The medical capsule 20 is formed from a filled casing made of a material which is dissolved by the digestive secretions of the intestinal tract. This casing includes a lower portion 26 and an upper portion 27 which are hermetically joined to each other. The wall thickness of the casing at both ends is designed to thin by shaping the interior (shaped portion 28). The filled casing is filled with the same material as the filling 15.

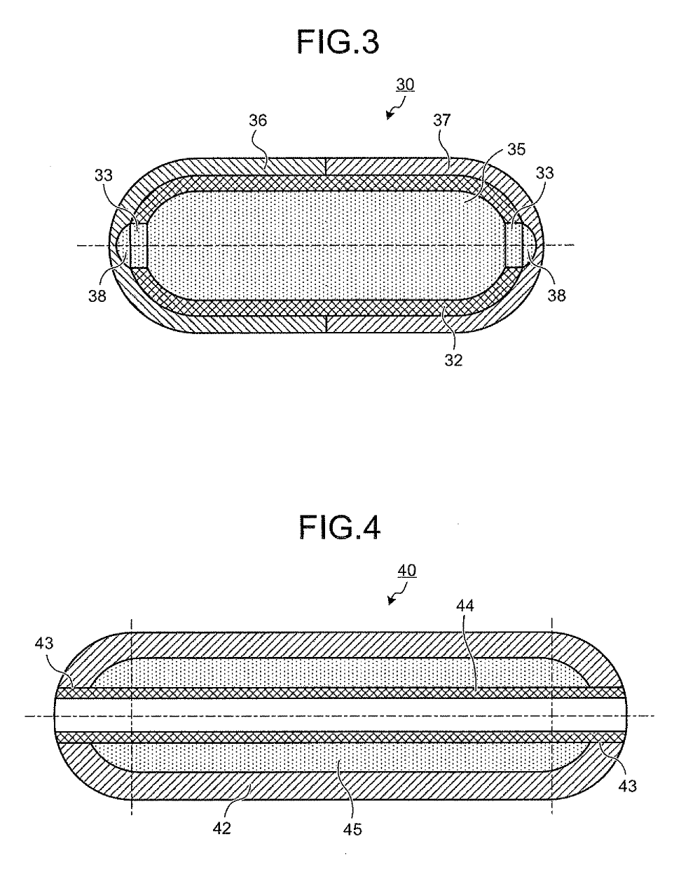

FIG. 3 shows a medical capsule 30 for testing patency of the intestinal tract. The medical capsule 30 includes an inner casing 32 with two discharge outlets (openings) facing each other and an outer casing formed by two cup-shaped members 36 and 37. The inner and outer casings 32 are made of a material which is dissolved by digestive secretions of the intestinal tract. The inner casing 32 is filled with a filling 35 made of the same material as the filling 15. The wall thickness of the outer casing at both ends may be designed to thin by shaping the interior (shaped portion 38) as shown in FIG. 1.

The medical capsule according to the first embodiment can assess whether a narrow area exists in the intestinal tract by a reliable method. This is achieved by the capsule, which is formed from the inner casing and the outer casing which are made of a material which is completely dissolved after a predetermined time by the digestive secretions of the intestinal tract. The inner casing is filled with a filling. The filling can flow into the intestinal tract through two facing discharge outlets after the outer casing dissolves.

However, there is a problem that the medical capsule can completely plug the narrow-area when reaching a narrow area in the intestinal tract. This may result in flatulence, so that the capsule cannot be excreted naturally. Or there may be an accumulation of liquid as a filling in a narrow area, so that the capsule cannot be excreted. These cases may result in emergency surgery.

A medical capsule according to a second embodiment is for testing patency of an intestinal tract, and it includes a discharge path or through-bore which is useful for ensuring flow path to certainly find and specify a narrow area in the intestinal tract.

A basic concept of the second embodiment is to provide a medical capsule which includes a hollow body made of a material which is dissolved after a predetermined time by the digestive secretions of the intestinal tract, and the medical capsule includes bores at both ends and a contrast medium.

A continuous tube made of the same material as the hollow body is inserted into the bores. The continuous tube includes no contrast medium and closes the hollow body. The continuous tube also includes a thinner wall than the hollow body and, therefore, dissolves earlier than the hollow body at a narrow area of the intestinal tract.

A space filled with a filling exists between an outer diameter of the continuous tube and an inner diameter of the hollow body. After dissolution of the tube by the intestinal secretions, the filling flows into the intestinal tract, resulting in a signal to the patient or an external device. However, the hollow body remains at the narrow area for a predetermined time required to completely dissolve the hollow body. The position of the narrow area can be detected by X-ray technology with a contrast medium contained in the hollow body.

A low-cost example of the medical capsule according to the second embodiment is one formed by a hollow body having only two facing bores without a tube or a filling. The hollow body dissolves after a predetermined time by the secretions of the intestinal tract, and contains a contrast medium. This medical capsule does not generate the above-mentioned signal at the narrow area. The presence of a narrow area in the intestinal tract is determined whether a medical capsule is contained in the stool extracted after an average time. Visibility of the medical capsule in the stool is improved if the medical capsule has a unique colour. If the medical capsule is not excreted after a pre-given time, its position in the intestinal tract can be determined because of the contrast medium by X-ray.

Another example of the medical capsule according to the second embodiment is one formed by a body having a penetrating slender bore (through-bore). This body dissolves by intestinal secretions after a pre-given time. Also a contrast medium may be added in the material.

The example of the second embodiment is explained in detail below with reference to FIGS. 4 to 6. FIG. 4 shows a medical capsule 40 for testing patency of the intestinal tract. The medical capsule 40 includes a hollow body 42 made of a material which is dissolved by secretions of the intestinal tract. The hollow body 42 has bores 43 at both ends and a through-bore formed with the bore 43. The hollow body 42 contains a contrast medium as a material. A continuous tube 44 is made of a material which is dissolved by the secretions of the intestinal tract, and is inserted into the bores 43. The continuous tube 44 has a thin wall which seals a hollow portion of the hollow body 42. A space 45 is formed between the continuous tube 44 and the hollow body 42, and is filled with a filling. The filling flows into the intestinal tract after the continuous tube 44 dissolves.

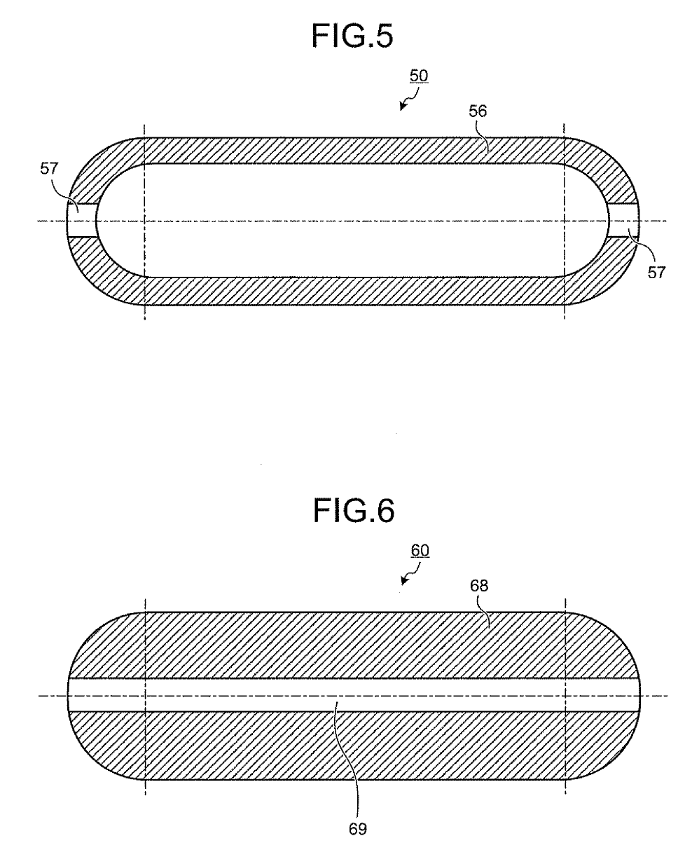

FIG. 5 shows a medical capsule 50 including a hollow body 56 having two facing bores 57. The hollow body 56 includes a material which is dissolved by the secretions of the intestinal tract, and contains a contrast medium.

FIG. 6 shows a medical capsule 60 includes a body 68 with a slender bore 69. The body 68 is made of a material which is dissolved by the secretions of the intestinal tract, and contains a contrast medium.

A medical capsule according to a third embodiment is explained below. A basic concept of the third embodiment is to provide a medical capsule which includes a hollow body made of a material which is dissolved after a predetermined time by the digestive secretions of the intestinal tract. The hollow body has bores at both ends and contains a contrast medium. The bores are closed with plugs (promptly-dissolving portion) which are resistant to the secretion of the stomach. The plugs are dissolved, for example, in six to eight hours after the medical capsule pass the stomach. The medical capsule is filled with liquid dye or powder dye. The liquid dye or the powder dye flows out of the medical capsule into the medium gastrointestine after the plugs dissolved, and colours an inner wall or the mucosa of the small intestine. The plug can be also one plug having such a shape as to close over the entire length of the through bore formed by the bores or having a pipe-shape.

The hollow body is designed to completely dissolve by the secretions of the intestine within 30 to 40 hours. If the hollow body is found in the stool, it will be determined that a gastrointestinal tract has enough patency to administer a video capsule. On the other hand, if the capsule does not show up in the stool after 20 to 25 hours, whether the capsule stuck at a narrow-area can be examined by X-ray.

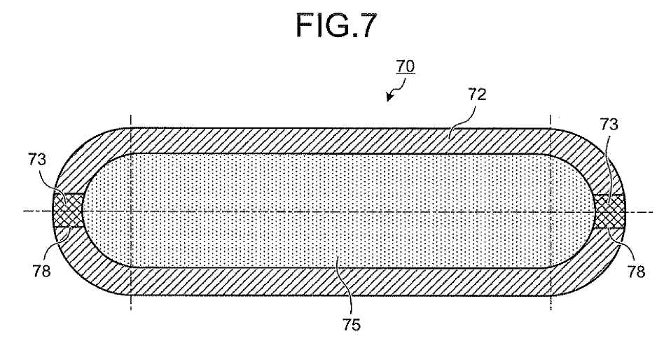

An example of the third embodiment is explained below with reference to FIG. 7. FIG. 7 shows a medical capsule 70 for colouring the medium gastrointestinal tract. The medical capsule 70 includes a hollow body 72 made of a material which is dissolved by the secretions of an intestinal tract. The hollow body 72 has bores 78 at both ends. The hollow body 72 also contains a contrast medium. Each bore 78 is closed with a plug 73. The plug 73 is made of a material which is resistant to the secretions of the stomach; however, it dissolves by the secretions of the intestine tract within approximately six to eight hours. The medical capsule 70 is filled with the above-mentioned filling such as a liquid dye 75, a powder dyer or the like. The filling flows into the middle gastrointestinal tract after the plug 73 dissolves.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A medical capsule, having a capsule shape and size capable of being introduced into a subject body, at least a part of the medical capsule being dissolved over time by remaining in a predetermined time or more in a lumen, the medical capsule comprising a casing, for forming the capsule shape, having at least two thin portions,
wherein a communication opening for communicatively connecting an inner space of the casing to an outside of the medical capsule is formed when the thin portion is eliminated earlier than other portions of the medical capsule by dissolution of the casing.

2. The medical capsule according to claim 1, wherein the casing is formed by an inner casing with a plurality of discharge outlets and an outer casing with a substantially uniform thickness, the outer casing surrounding an outer surface of the inner casing.

3. The medical capsule according to claim 1, wherein the thin portion is formed so that an interior of a wall of the casing is shaped at least two portions with a substantially uniform thickness.

4. The medical capsule according to claim 1, wherein the casing is formed by an inner casing with a plurality of discharge outlets and an outer casing with a substantially uniform thickness, the outer casing surrounding an outer surface of the inner casing, and
the thin portion is formed so that an interior of a wall of the casing is shaped at positions facing the discharge outlets of the outer casing.

5. The medical capsule according to claim 1, wherein the casing is formed by two cup-shaped members which are connected to each other.

6. The medical capsule according to claim 1, wherein the casing contains a contrast medium visible by X-ray as a material.

7. The medical capsule according to claim 6, wherein the contrast medium is homogeneously-mixed and added to the material of the casing.

8. The medical capsule according to claim 6, wherein the contrast medium is barium sulphate.

9. The medical capsule according to claim 1, wherein the space is substantially similar to an outline of the casing.

10. The medical capsule according to claim 1, wherein the space is filled with a filling of at least one of granulated material, powder, gel and liquid.

11. The medical capsule according to claim 1, wherein the space is filled with a filling of at least one of gas with an exceptionally striking smell, gel, liquid dye, painkiller, and other medicine.

12. A medical capsule, having a capsule shape and size capable of being introduced into a subject body, at least a part of the medical capsule being dissolved over time by remaining in a predetermined time or more in a lumen, the medical capsule comprising
a casing, for forming the capsule shape, having a thin portion; and
a filling with which an inner space of the casing is filled,
wherein the filling is discharged to an outside of the medical capsule after dissolution of the thin portion, with an portion of the casing other than the thin portion keeping substantially the capsule shape.

13. The medical capsule according to claim 12, wherein the thin portion includes a plurality of thin portions provided at different positions, respectively.

14. The medical capsule according to claim 13, wherein the thin portions are provided at facing positions corresponding respectively to the front and back of the medical capsule.

15. The medical capsule according to claim 12, wherein the casing is formed by an inner casing with a plurality of discharge outlets and an outer casing with a substantially uniform thickness, the outer casing surrounding an outer surface of the inner casing.

16. The medical capsule according to claim 12, wherein the thin portion is formed so that an interior of a wall of the casing is shaped at least two portions with a substantially uniform thickness.

17. The medical capsule according to claim 12, wherein the casing is formed by an inner casing with a plurality of discharge outlets and an outer casing with a substantially uniform thickness, the outer casing surrounding an outer surface of the inner casing, and
the thin portion is formed so that an interior of a wall of the casing is shaped at positions facing the discharge outlets of the outer casing.

18. The medical capsule according to claim 12, wherein the casing is formed by two cup-shaped members which are connected to each other.

19. The medical capsule according to claim 12, wherein the casing contains a contrast medium visible by X-ray as a material.

20. The medical capsule according to claim 19, wherein the contrast medium is homogeneously-mixed and added to the material of the inner casing.

21. The medical capsule according to claim 19, wherein the contrast medium is barium sulphate.

22. The medical capsule according to claim 12, wherein the space is substantially similar to an outline of the casing.

23. The medical capsule according to claim 12, wherein the filling is at least one of granulated material, powder, gel and liquid.

24. The medical capsule according to claim 12, wherein the filling is at least one of gas with an exceptionally striking smell, gel, liquid dye, painkiller, and other medicine.

25. A method for testing patency of an intestinal tract with a medical capsule having a capsule shape and size capable of being introduced into a subject body, at least a part of the medical capsule being dissolved over time by remaining in a predetermined time or more in a lumen, the method comprising
using as the medical capsule a capsule in which at least two thin portions of a casing forming the capsule shape is dissolved after a predetermined time to form a communication opening for communicatively connecting an inner space of the casing to an outside of the medical capsule, and the capsule keeps the capsule shape further after a predetermined time.

26. The method according to claim 25, wherein the casing contains a contrast medium as a material, the method comprising detecting the medical capsule by X-ray.

27. The method according to claim 25, wherein the filling is at least one of granulated material, powder, gel and liquid.

28. A method for testing patency of an intestinal tract with a medical capsule having a capsule shape and size capable of being introduced into a subject body, at least a part of the medical capsule being dissolved over time by remaining in a predetermined time or more in a lumen, the method comprising
using as the medical capsule a capsule,
the capsule including a casing, for forming the capsule shape, having a thin portion; and
a filling with which an inner space of the casing is filled, wherein the filling is discharged to an outside of the capsule after dissolution of the thin portion, with an portion of the casing other than the thin portion keeping substantially the capsule shape.

29. The method according to claim 28, wherein the casing contains a contrast medium as a material, the method comprising detecting the medical capsule by X-ray.

30. The method according to claim 28, wherein the filling is at least one of granulated material, powder, gel and liquid.

31. The method according to claim 26, wherein the filling is at least one of gas with an exceptionally striking smell, gel, liquid dye, painkiller, and other medicine.

32. A method for treating an intestinal tract with a medical capsule having a capsule shape and size capable of being introduced into a subject body, at least a part of the medical capsule being dissolved over time by remaining in a predetermined time or more in a lumen, the method comprising
using as the medical capsule a capsule,
the capsule including a casing, for forming the capsule shape, having a thin portion; and
a filling with which an inner space of the casing is filled, wherein the filling is discharged to an outside of the capsule after dissolution of the thin portion, with an portion of the casing other than the thin portion keeping substantially the capsule shape.

33. The method according to claim 32, wherein the casing contains a contrast medium as a material, the method comprising detecting the medical capsule by X-ray.

34. The method according to claim 32, wherein the filling is at least one of granulated material, powder, gel and liquid.

35. The method according to claim 32, wherein the filling is at least one of gas with an exceptionally striking smell, gel, liquid dye, painkiller, and other medicine.

36. A medical capsule, having a capsule shape and size capable of being introduced into a subject body, at least a part of the medical capsule being dissolved over time by remaining in a predetermined time or more in a lumen, the medical capsule comprising a casing, for forming the capsule shape, having an promptly-dissolving portion,
wherein a communication opening for communicatively connecting an inner space of the casing to an outside of the medical capsule is formed when the promptly-dissolving portion is eliminated earlier than other portions of the promptly-dissolving portion by dissolution of the promptly-dissolving portion.

37. The medical capsule according to claim 36, wherein the promptly-dissolving portion is a thin portion of the casing.

38. The medical capsule according to claim 36, wherein the promptly-dissolving portion is a plug for closing a bore of the casing.

39. The medical capsule according to claim 36, wherein the promptly-dissolving portion is resistant to secretion of a stomach and is made of a material which dissolves by the secretion of the stomach earlier than the casing.

40. The medical capsule according to claim 36, wherein the promptly-dissolving portion is a plug for closing over an entire length of a through-bore across the casing.

41. The medical capsule according to claim 40, wherein the plug has a pipe-shape, and the plug is resistant to secretion of a stomach and is made of a material which dissolves by the secretion of the stomach earlier than the casing.

42. The medical capsule according to claim 36, wherein the casing contains a contrast medium visible by X-ray as a material.

43. The medical capsule according to claim 36, wherein the space is substantially similar to an outline of the casing.

44. The medical capsule according to claim 36, wherein the space is filled with a filling of at least one of granulated material, powder, gel and liquid.

45. The medical capsule according to claim 36, wherein the space is filled with a filling of at least one of gas with an exceptionally striking smell, gel, liquid dye, painkiller, and other medicine.

46. A medical capsule for testing patency of an intestinal tract or treating an intestinal tract, the medical capsule comprising:
a first state of dimension such that the capsule passes through an intestinal tract having no narrow area but is stopped by a narrow area;
a second state of dimension such that the capsule can pass the narrow area of the intestinal tract; and
an indicator material covered by a portion of the capsule hindering the indicator material to flow into the intestine in the first state and flows into the intestine in the second state,
wherein the portion of the capsule is dissolvable by secretions of the intestine after a predetermined time to allow the indicator material to flow into the intestine.

47. The medical capsule according to claim 46, wherein the indicator material includes at least one of a dye, a foam, and a substance producing striking smell.

48. The medical capsule according to claim 46, wherein the portion covering the indicator material is a layer which partially covers the medical capsule.

49. The medical capsule according to claim 46, wherein the portion covering the indicator material is a plug of a wall of the medical capsule.

50. The medical capsule according to claim 46, the predetermined time is longer than the time needed for the medical capsule to pass an intestinal tract having no narrow area which hinders the medical capsule to pass.

51. The medical capsule according to claim 46, wherein the first state of dimension has a dimension corresponding to the dimension of a video capsule for imaging the intestine tract.

52. The medical capsule according to claim 46, comprising a path allowing passage of liquid or gas through the medical capsule.

53. The medical capsule according to claim 46, wherein the portion hindering the indicator material to flow out the capsule in the first state is not covered by other portion of the medical capsule and is not permeable with regard to intestine secretions.

54. The capsule according to claim 46, wherein the dissolvable portion of the medical capsule is an outer layer covering an inner layer that includes at least a communication opening,
the communication opening, after removal of the outer layer, is communicatively connected to a surrounding area of the medical capsule in an inner space of the capsule, and
the inner space contains the indicator material so that the indicator material can flow out of the capsule through the communication opening.

55. The medical capsule according to claim 54, comprising at least two communication openings at both sides of the medical capsule.

56. The capsule according to claim 54, wherein the inner layer is dissolve after a second predetermined time longer than the predetermined time.

57. The capsule according to claim 46, comprising an X-ray contrast medium.

58. The capsule according to claim 46, wherein the dissolvable portion is an outer layer of the medical capsule,
the outer layer having at least a thin portion which provides a communication opening that communicatively connects an inner space of the capsule to a surrounding area of the capsule after dissolution of a portion of the outer layer, and
the inner space contains the indicator material so that the indicator material can flow out of the medical capsule.

59. A medical capsule for coloring an intestinal wall, comprising:
a portion which is non-dissolvable by secretions of a stomach but which is dissolvable by secretions of an intestine after a predetermined time; and
a dye covered by the portion in the medical capsule, not flowing out of the capsule when the portion is not dissolved but flowing out of the capsule when the portion is dissolved.

60. A system comprising:
a video capsule for imaging an intestinal tract; and
a medical capsule for testing patency of an intestinal tract according to claim 1.

61. A system comprising:
a video capsule for imaging an intestinal tract; and
a medical capsule for testing patency of an intestinal tract according to claim 12.

62. A system comprising:
a video capsule for imaging an intestinal tract; and
a medical capsule for testing patency of an intestinal tract according to claim 31.

63. A system comprising:
a video capsule for imaging an intestinal tract; and
a medical capsule for testing patency of an intestinal tract according to claim 41.

64. A system comprising:
a video capsule for imaging an intestinal tract; and
a medical capsule for testing patency of an intestinal tract according to claim 54.
